(19) United States
(12) Patent Application Publication
(10) Pub. No.: US 2025/0268643 A1
(43) Pub. Date: Aug. 28, 2025

(54) ELECTROSURGICAL INSTRUMENT WITH CARBON FIBER ELECTRODE

(71) Applicant: Erbe Elektromedizin GmbH, Tuebingen (DE)

(72) Inventors: Uwe SCHNITZLER, Tuebingen (DE); Daniel SADLER, Tuebingen (DE)

(21) Appl. No.: 19/051,828

(22) Filed: Feb. 12, 2025

(30) Foreign Application Priority Data
Feb. 23, 2024 (EP) 24159496.9

(51) Int. Cl. A61B 18/04; A61B 18/00

(52) U.S. Cl. CPC A61B 18/042; A61B 2018/00077; A61B 2018/00095; A61B 2018/00178; A61B 2018/00184

(57) ABSTRACT

A surgical instrument for the treatment of a tissue surface, particularly for coagulation or ablation of the latter, an electrode consisting of carbon fibers or at least comprising carbon fibers for current conduction to the tissue surface. Anisotropy of the electrical conductance of the carbon fibers or the formed electrode results in a large area and uniform current distribution. Due to the configuration of the electrode surface by carbon fibers and particularly their high thermal conductivity, a sticking of the electrode to the tissue surface is effectively avoided.

This application claims priority to European Patent Application No. 24159496.9 filed Feb. 23, 2024, the entirety of which is incorporated herein.

The invention is directed to an instrument for electro-surgical treatment of living tissue of human or animal patients. Particularly, the invention refers to an instrument for superficial coagulation of such tissue.

For treatment of living tissue, particularly coagulation and/or ablation, instruments for argon plasma coagulation are known. Such an instrument is disclosed in WO 2021/013852 A1, for example. The instrument disclosed there is configured as flexible probe and comprises a hose having a gas-conveying lumen. In the proximity of the distal gas outlet opening of the hose an electrode is arranged, which is connected via an electrical supply line with a generator that supplies a voltage to the electrode sufficiently high for plasma generation. The argon stream flowing through the lumen is ionized at the electrode and exits as plasma jet in distal direction from the instrument.

With such an instrument selected tissue areas can be treated precisely and distinctly.

However, occasionally a treatment of larger tissue areas is desired, for example during mucosa ablation in the stomach. There, it can be necessary to treat larger areas, for example up to two-thirds of the stomach surface, electrosurgically by means of argon plasma coagulation.

For this EP 3 141 203 B1 and EP 3 141 204 B1 disclose an ablation instrument respectively that is suitable for extensive mucosa ablation. The instrument comprises a probe hose supporting a head at its distal end in which the lumen of the hose is first continued and then forked. The lumen opens out in this manner in two outlet openings separated from one another. Therein, electrodes are arranged that produce a plasma jet in each case. The two created plasma jets impinge next to one another on the tissue surface. In this manner a wider ablation stripe is created.

Another instrument for argon plasma coagulation is known from DE 195 35 811 C1. This instrument comprises a tube in the end of which a nozzle is inserted. In a variant, the nozzle comprises a block of sintered small balls made of insulating ceramic material or also of electrically conductive material, such as metal or carbon, at its exit opening. In this case it is recommended to insert the nozzle additionally in an insulating sleeve in order to avoid a direct contact with the tissue.

Moreover, instruments for contacting tissue coagulation are known, in which a coagulation electrode is brought into direct contact with living tissue. For this purpose, U.S. Pat. No. 4,074,718 B1 discloses an instrument having a carbon-coated electrode. The carbon coating shall avoid sticking of the electrode to the tissue.

Also, US 2013/0110105 A1 discloses an electrosurgical instrument having a distal electrode that is provided with a plastic coating.

Further prior art is known from WO 2014/197632 A2, CN 11 683 08 A, US 2012/0083782 A1, US 2016/0121134 A1 and EP 3 708 222 A1.

While depending on the diameter and the size of the plasma jet, a distinct area of the tissue can be treated, the contact coagulation allows an even more specific local treatment of a sharply limited area.

It is the object of the invention to provide an electrosurgical instrument by which extensive surface areas of biological tissue can be coagulated quickly and uniformly on the surface.

Embodiments of the invention include an instrument having a shank that comprises an electrode at its distal end, wherein the electrode can be supplied via a line and an electrical generator with treatment current. The electrode comprises carbon fibers that are electrically conductive. The carbon fibers cause the electrical conductivity of the electrode and concurrently provide an extensive current distribution if the electrode contacts a larger area of the biological tissue due to its own spatial extension. The electrode being attached to the oblong shank can thereby have a width, particularly measured transverse to the shank longitudinal direction that is larger than the width of the shank. The carbon fibers thereby form individual line-shaped conductors having a resistance, which individually conduct electrical current and distribute the electrical current relatively uniformly on the surface on which a contact between the electrode and the tissue is established.

The longitudinal shank can be a stiff or flexible shank that supports the electrode at its distal end and comprises a connector for connection of the electrical line to a generator at its proximal end. The carbon fibers comprising electrode serves predominantly for contact coagulation in this case.

It is also possible to provide one or more lumens in the longitudinal shank so that the shank is configured as hose or tube. The lumen can be connected or is connected to a gas supply source. If multiple lumen are present, they can be connected to one and the same gas source or also to different gas sources.

On the distal end of the hose (or tube) an outlet opening can be provided that is connected with the lumen or the lumens. A suitable gas can flow out of the outlet opening, particularly an inert gas such as argon. The outlet opening and the electrode are preferably arranged relative to one another, so that the ejected gas flows around the electrode.

The electrode can be supported in or on the shank immovably or movably. If the shank is configured as hose or tube, the electrode can be arranged completely or partly inside the lumen or outside the lumen, for example in front of the outlet opening. Particularly the electrode can be arranged in a manner to be movable out of the lumen. The electrode can serve for ionization of a gas flow and thus for plasma creation as well as concurrently or alternatively for contact coagulation of the tissue surface to be treated. Particularly, if the electrode comprises an ohmic resistance that is higher than the treatment voltage divided through the maximum treatment current, the electrode has a current limiting effect for the current used for contact coagulation and therefore furthermore allows the creation of plasma for coagulation of tissue. This can contribute to achieve a uniform and extensive and also quick coagulation of the tissue.

The electrode consisting of or comprising carbon fibers can additionally have a preferred direction for the current conduction, wherein the preferred direction is defined by the carbon fibers. This can also result in an equalization of the current distribution over the entire electrode. For example, the electrode can have a low electrical resistance in electrode longitudinal direction. This can be supported by a metal wire extending in electrode longitudinal direction in the electrode, for example, centered in the electrode. Originating from the metal wire, the current has to overcome an electrical resistance on its way to the electrode surface, which can contribute to avoiding or reducing local current density peaks on the electrode surface.

The electrode can be arranged in or at the outlet opening of the lumen in movable manner. For example, it can be arranged in a first position inside the lumen, whereas in a second position it is located completely or partly outside the lumen. In this manner, the instrument can prefer different coagulation types depending on the position of the electrode. For example, the instrument can serve for plasma coagulation as long as the electrode is positioned inside the lumen. However, if the electrode is entirely or partly outside the lumen, a contact coagulation can complement the plasma coagulation or, in case of large area tissue contact of the electrode, the contact coagulation can be predominantly or solely effective.

The electrode can be configured rigidly or flexibly, for example as brush, loop, twine or rigid or flexible spatula. The carbon fibers can be embedded into a solid body, for example into a plastic matrix. The plastic matrix can be made from electrically insulating plastic. Thereby the number and arrangement of carbon fibers determine the conductivity and the preferred current flow direction in the electrode as well as finally also the current distribution on the electrode surface. It is, however, also possible to use an intrinsically or extrinsically conductive plastic for the plastic matrix.

The carbon fibers can be entirely embedded into the body of the electrode or can have ends projecting out of the body. Likewise, the carbon fibers can be connected together by braiding, twining or other measures to form a thread or twine, which is connected at one end or at its both ends with the current supply line. It is also possible to hold all of the carbon fibers at one end respectively in a holder, while the other ends project in the type of a brush out of this holder and away therefrom. It is possible to arrange, in addition to the carbon fibers, other electrical conductors in the electrode, for example a blank metal wire or the like, to specifically influence the spring characteristic of a loop-formed electrode or the electrical characteristic thereof.

Moreover, it is possible to configure the flexibility of the electrode, so that the electrode fits in compressed condition in the lumen of the hose on one hand and, if it is positioned outside the lumen, on the other hand expands and comprises a transverse dimension in this way that is larger than the transverse dimension of the lumen.

Further details of advantageous embodiments of the invention are subject matter of claims as well as the drawing and the respective description. The drawings show:

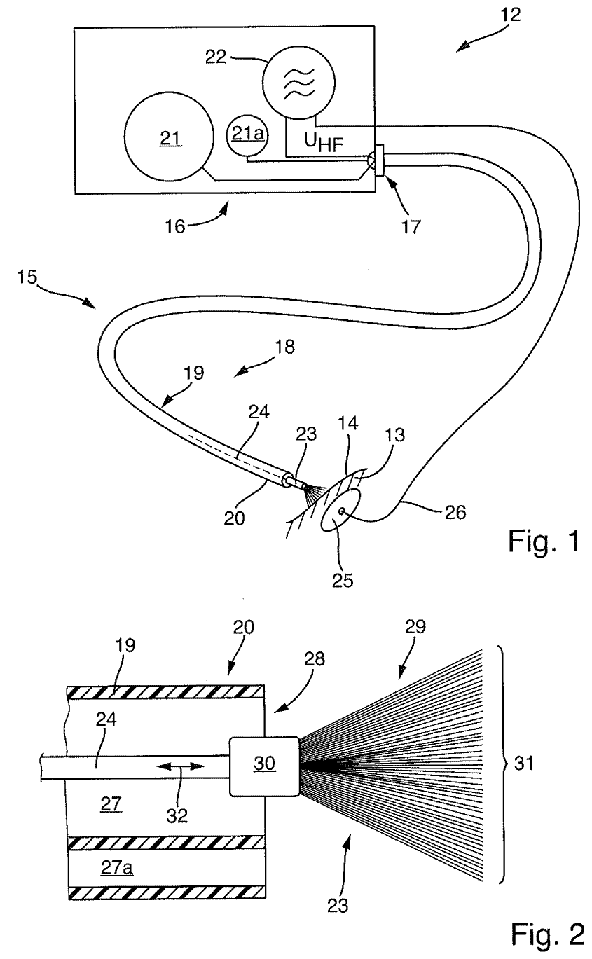

FIG. 1 illustrates a first embodiment having an apparatus for supply of an instrument as well as biological tissue that is subject to a thermal treatment in schematic illustration,

FIG. 2 illustrates the distal end of the instrument according to FIG. 1,

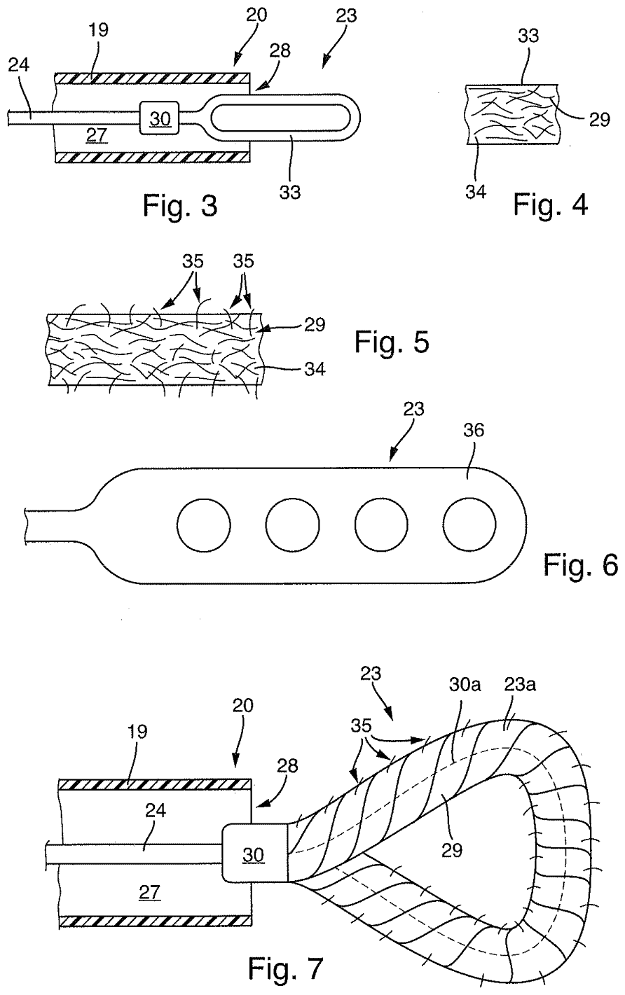

FIG. 3 illustrates the distal end of an alternative embodiment of an instrument according to the invention in longitudinally cut illustration,

FIG. 4 illustrates a portion of the electrode of the instrument according to FIG. 3,

FIG. 5 illustrates a portion of an alternative embodiment of the electrode of the instrument according to FIG. 3,

FIG. 6 illustrates an alternative electrode form for the instrument according to FIG. 3 having a material structure according to FIG. 4 or 5,

FIG. 7 illustrates the distal end of the instrument according to FIG. 1 in an alternative configuration having an electrode formed as twine in form of a loop,

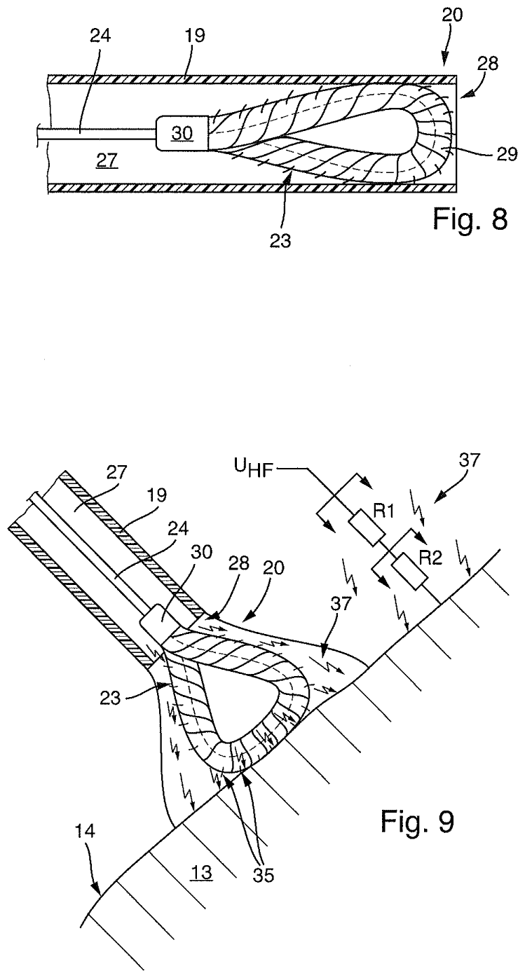

FIG. 8 illustrates the instrument according to FIG. 7 with the electrode being retracted inside the lumen of the shank,

FIG. 9 illustrates the electrode according to FIG. 7 during coagulation of biological tissue and an additional schematic simplified illustration of the electrical conditions,

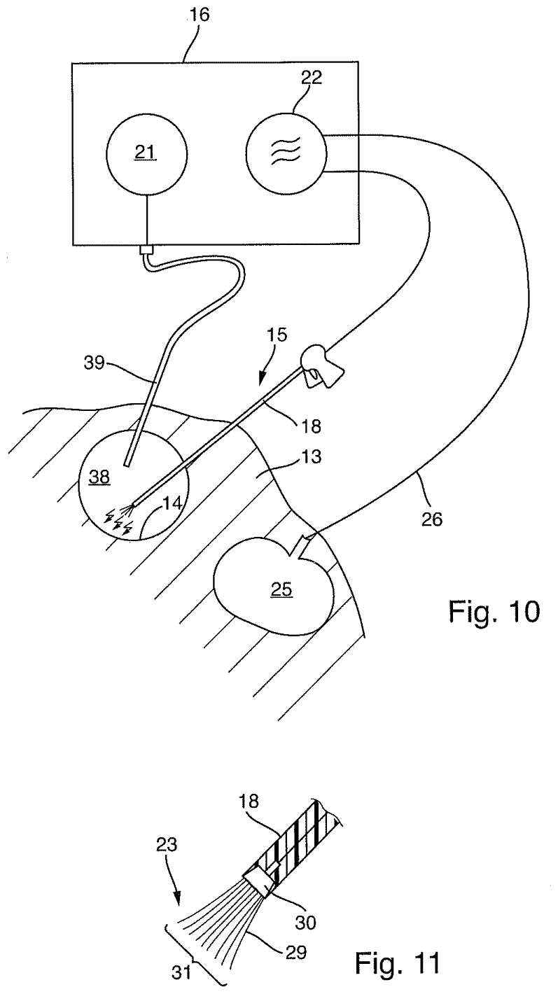

FIG. 10 illustrates an alternative embodiment of the instrument according to the invention without its own gas supply,

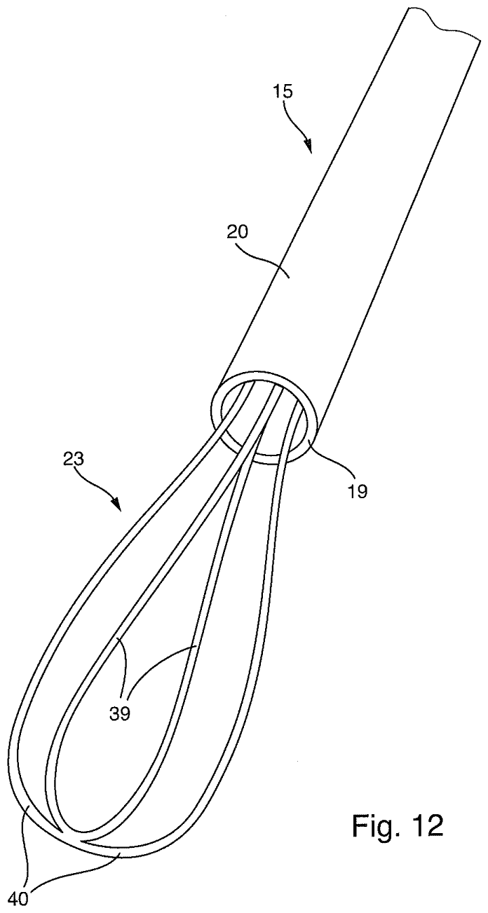

FIG. 11 illustrates the distal end of the instrument according to FIG. 10 in longitudinally cut illustration and

FIG. 12 illustrates the distal end of a modified instrument in an alternative configuration having an electrode in double-loop shape.

FIG. 1 shows a treatment device 12 serving for two-dimensional coagulation of biological tissue 13. The biological tissue 13 comprises a surface 14 (see also FIG. 9) that can be formed from an interior organ surface, such as the gastric mucosa or the like.

An instrument 15 and an apparatus 16 configured for supply of the instrument 15 are part of the device 12, wherein connector 17 provides a connection at the proximal end of the instrument 15 to the apparatus 16.

The instrument 15 comprises a long slim shank 18 that is configured in the present case as flexible hose 19 or also as tube. The flexible hose 19 is thereby configured to be brought through the working channel of a not illustrated endoscope or another instrument providing access to the body interior of a patient to the treatment location opposed to the tissue surface 14. While the proximal end of hose 19 is connected to the apparatus 16 outside of the patient, the distal end 20 of instrument 15 is located on or inside the patient.

The instrument 15 illustrated in FIG. 1 serves for coagulation of the tissue surface 14 under an argon plasma atmosphere. For this purpose, apparatus 16 comprises a gas source 21 that is configured to supply the instrument 15 via connector 17 with argon or also another suitable gas. Moreover, the apparatus 16 comprises a high frequency generator 22 that is connected with one pole to a line 24 leading to an electrode 23 of the instrument 15 and with its other pole to a line 26 leading to a neutral electrode 25. The neutral electrode 25 is to be extensively attached on the patient.

As illustrated in FIG. 2, hose 19 encloses at least one lumen 27 via which the gas, preferably argon, supplied to the hose 19, flows toward electrode 23 and flows out of an outlet opening 28 arranged at the distal end 20 of hose 19. Optionally, hose 19 can comprise one or more additional lumens 27a that can be connected to the same gas source 21 or to one or more additional fluid sources 27a, particularly gas sources or also liquid sources.

The electrode 23 comprises a multiplicity of carbon fibers 29 that can be arranged, for example, in the type of a brush. For this purpose, they comprise an end held in a holder 30 and extend from the holder 30 in distal direction relative to the holder 30 and thus away from the opening 28. As indicated in FIG. 2, thereby, the carbon fibers 29 can be arranged to project in divergent manner from holder 30, so that the so formed brush 31 can have a width exceeding the diameter of lumen 27. The width is thereby to be measured transverse to the lumen and thus transverse to the line 24. The brush 31 conducts electrical current nearly exclusively in fiber longitudinal direction, whereby each fiber 29 has an electrical resistance. Thereby a contact of one or a few carbon fibers with the tissue surface 14 does not result in that the total current supplied from the generator flows via these fibers. Rather the generator voltage is maintained at the remaining carbon fibers.

The electrode 23 can be axially immovably arranged or also movably arranged in longitudinal direction of the lumen and thus in longitudinal direction of line 24. For example, electrode 23 can be moved into lumen 27 or out of the latter by a respective positioning along arrow 32 (FIG. 2).

The electrode 23 of instrument 15 can be configured differently. For this purpose, FIG. 3 illustrates an embodiment in which the electrode 23 is configured as oval body 33 being rigid or having low flexibility. The body 33 is preferably a plastic body, the inner structure of which is apparent from FIG. 4. The plastic body is formed by a plastic matrix 34 made from electrically low-conducting or non-conducting plastic in which numerous carbon fibers are embedded. These can be relatively short fibers as well as longer fibers. The carbon fibers 29 can have a preferred direction or can be arranged alternatively without preferred direction. The carbon fibers can be completely embedded in the body 33, so that the body 33 has a smooth surface. Alternatively, as shown in FIG. 5, the carbon fibers 29 can have ends 35 projecting from the plastic matrix 34 so that the electrode then has a rough or hairy surface.

The electrode 23 is by no means limited to the oval shape illustrated in FIG. 3. It can also be configured as lancet-shaped or oval spatula, as needle or rod or as perforated spatula 36 as illustrated in FIG. 6.

In another modified embodiment illustrated in FIG. 7, carbon fibers 28 of the electrode 23 are formed to a rope or twine 23a, the two ends of which are held in the holder 30. Thus, electrode 23 forms a flexible, smooth sling or loop. It comprises carbon fibers that are twined or twisted with one another, whereby the ends 35 thereof can project out of twine 23a or the rope. If desired, twine 23a can comprise a wire 30a made of metal or plastic arranged approximately centered in the twine 23a, which is symbolized by a dashed line in FIG. 7. This (plastic) wire 30a can serve to stiffen twine 23a. If it is made of metal or if the plastic wire is combined with a metal wire, it can additionally serve to provide uniformity of current distribution.

The electrode 23 formed by twine 23a or the loop can be held on the shank 18 in a predefined fixed position. It is, however, also possible to movably arrange the electrode 23. In this case, the electrode 23 can be brought either into an exposed position illustrated in FIG. 7 or into a retracted position illustrated in FIG. 8 by a respective axial movement. In the retracted position, electrode 23 is partly or entirely held inside lumen 27. The electrode 23, exemplarily formed as loop, can have a width in the extended condition according to FIG. 7 that is larger than the inner diameter of hose 19 and thus lumen 27. Thanks to its flexibility, electrode 23 can be compressed with regard to its width, so that it fits into lumen 27 according to FIG. 8.

The function of the instrument 15 described so far is explained in the following by way of example and with reference to FIG. 9.

For coagulation of the tissue surface 14, instrument 15 is brought with its distal end 20 in proximity to the tissue surface 14. Argon flows via lumen 27 in distal direction and flows around electrode 23. The latter is connected via the line 24 to the high frequency generator 22 and receives therefrom an alternating voltage of multiple hundred Volts having a frequency of remarkably more than 100 kHz. The electrode 23 can thereby be positioned projecting out of opening 28 and at least partly selectively touch the tissue surface 14. However, it comprises a resistance per unit length along its length that is indicated in FIG. 9 by accompanying resistances R1, R2 symbolically where the voltage UHF applies, which is output by generator 22.

Due to the direct contact to the tissue surface 14, the electrode 23 can there produce a coagulation effect. Thereby argon flowing through lumen 27 can surround the electrode 23 in protective manner and result in that a spark plasma is formed that is in contact with the tissue surface 14 and that originates from electrode 23. Depending on the size of the contact area between electrode 23 and the tissue surface 14 and the pressure, the electrical resistance formed by electrode 23 between line 24 and tissue surface 14 varies to higher or lower values. Precisely due to the non-metallic configuration of electrode 23, the current transferred from electrode 23 into the tissue 13 at the contact positions can be limited, so that in addition to the contact coagulation a plasma creation takes place, for example at the holder 30 or at parts of the electrode 23. The plasma is illustrated in FIG. 9 in the physical illustration as well as in the accompanying schematic electrical illustration by corrugated arrows 37.

The carbon fibers 29 can contribute to a strong electrical anisotropy of electrode 23. For example, the ends 35 of the carbon fibers projecting from twine 23a or also from the electrodes according to FIG. 3 or 6 can serve as preferred current exit points from which plasma threads originate. However, particularly the configuration of electrode 23 made of carbon fibers 29 avoids adhesion or sticking of electrode 23 on the tissue surface 14 and thus an injury thereof. Whether the electrode 23 is configured as brush 31, as spatula 36 or as oval body 33—in any case the carbon fibers 29 contribute to the uniform current distribution on the tissue surface 14 and to avoid sticking of electrode 23 thereon. Additionally, the high thermal conductivity of the carbon fibers can be used to keep the temperature of the electrode relatively low, whereby also the tendency of the tissue to stick to the electrode can be reduced.

Another embodiment of the invention is derived from FIGS. 10 and 11.

In the embodiment according to FIG. 10, the instrument 15 comprises a rigid shank 18 that can comprise a gas-conveying lumen like the above embodiments. It is however also possible to omit a gas supply through the shank 18 and to supply the body cavity 38 via a separate access 39 with gas from the gas source. The electrode 23 can thereby be configured in any type described above. By way of example FIG. 11 illustrates the configuration as brush 31. Originating from the free ends of carbon fibers 29 plasma threads can be formed toward the tissue surface 14 if the body cavity 38 is filled with protective gas, for example argon. The treating person can now move the brush 31 with contact or without contact of the body tissue surface 14 over large areas thereof and coagulate the latter.

Numerous further variations are possible on the instrument. For example, FIG. 12 illustrates the instrument 15 largely according to the embodiments described in FIGS. 7 to 9. While, however, electrode 23 is configured as single loop in the embodiments according to FIGS. 7 to 9, it is possible to configure the electrode also with multiple loops, for example two loops 39, 40 that are positioned about 90° relative to each other. At its proximal end, this electrode 23 can be electrically connected to a conductor extending through the hose 19. The loops 39, 40 can in turn be configured as twines, twisted or braided threads from carbon fibers that are connected to the conductor extending through the hose 19, for example by crimping. In terms of the operation and function of instrument 15 according to FIG. 12, the above explanations apply accordingly using the already introduced reference signs respectively. One or more wires can be included within the loops 39, 40 in order to increase the stiffness of electrode 23 and to provide the latter with spring elastic recovery characteristics. Similar to the wire 30a in FIG. 7, these wires can extend centrally through the respective legs of the loops 39, 40.

In all of the above-described embodiments the electrode consisting of carbon can be electrically contacted by a supply line made of metal. It is particularly advantageous, if the element by which the electrodes are held, for example, a crimping sleeve and potentially also the electrical supply line have an increased thermal conductivity. For example, the supply line can be made of copper or it can also be made of a stainless steel wire provided with a thermally conductive coating.

In an instrument 15 according to the invention for surgical treatment of a tissue surface 14, particularly for coagulation or ablation of the latter, an electrode 23 consisting of carbon fibers 29 or at least comprising carbon fibers 29 serves for current conduction to the tissue surface 14. Due to the anisotropy of the electrical conductance of the carbon fibers 29 or the formed electrode 23, an extensive and uniform current distribution can be achieved. And this indeed, in the case of direct contact coagulation as well as in mixed coagulation during plasma creation under at least partial contact to the tissue surface 14 by the electrode 23. Due to the configuration of the electrode surface by carbon fibers 29 and particularly their high thermal conductivity, a sticking of the electrode 23 to the tissue surface 14 is effectively avoided.

1. An instrument for treating living tissue comprising:
a longitudinal shank having an electrode at one end, the electrode connected to an electrical line configured to be connected to an electrical generator,
wherein the electrode comprises carbon fibers.

2. The instrument according to claim 1, wherein the longitudinal shank is a hose or tube that comprises at least one lumen that is or can be connected to a gas source and that comprises an outlet opening at a distal end of the hose or tube.

3. The instrument according to claim 1, wherein the electrode is arranged in or at the outlet opening.

4. The instrument according to claim 1, wherein the electrode is moveably supported in or on the shank.

5. The instrument according to claim 1, wherein the electrode is configured to be entirely retractable into the lumen.

6. The instrument according to claim 1, wherein the electrode is configured as loop.

7. The instrument according to claim 1, wherein the electrode is configured in flexible manner.

8. The instrument according to claim 1, wherein at least some of the carbon fibers have a movable uncovered end.

9. The instrument according to claim 1, wherein at least some of the carbon fibers have an end that is fixed in the electrode.

10. The instrument according to claim 1, wherein the carbon fibers are held in the type of a brush.

11. The instrument according to claim 1, wherein the carbon fibers are configured as twine.

12. The instrument according to claim 1, wherein the electrical line is configured in a manner extending from the electrode up to a proximal connector.

13. The instrument according to claim 1, wherein the electrical line is arranged inside the lumen.

14. The instrument according to claim 1, wherein the shank comprises multiple lumens that lead from a proximal end of the shank to the distal end.

15. The instrument according to claim 14, wherein the lumens are connected with a connector that is configured for connection of the lumens with different fluid supply sources.
